(19) United States
(12) Patent Application Publication
(10) Pub. No.: US 2006/0229646 A1
(43) Pub. Date: Oct. 12, 2006

(54) Forward-directed atherectomy catheter

(76) Inventor: Kurt D. Sparks, Palo Alto, CA (US)

Correspondence Address: COURTNEY STANIFORD & GREGORY LLP, P.O. BOX 9686, SAN JOSE, CA, 95157, US

(21) Appl. No.: 11/104,902

(22) Filed: Apr. 12, 2005

(51) Int. Cl. A61B 17/22

(52) U.S. Cl. 606/159

(57) ABSTRACT

A catheter system is described for operation within a stenosed blood vessel. The catheter system includes a catheter shaft having at least one lumen. The catheter system further includes a convex distal housing that includes a series of openings along a convex surface that allow vascular plaque tissue to enter the interior of the distal housing. The catheter system also includes an internal rotational cutter having blades that are in proximity to the portion of the inner surface of the distal housing that includes the openings. Additionally, the catheter system includes a drive shaft coupled to the internal rotational cutter.

### FIELD OF THE INVENTION

This invention applies to the field of interventional cardiology and interventional radiology, and more specifically to describe interventional (catheter) based systems designed to establish patent pathways through vascular chronic total occlusions (CTOs) and to debulk, or remove diseased tissue, or commonly referred to as plaque from stenosed coronary and peripheral arteries and veins.

### BACKGROUND OF THE INVENTION

Cardiovascular and peripheral artery disease are routinely treated with interventional (catheter based) methods wherein balloon angioplasty and stenting re-establish patent blood flow to a vessel that has undergone the gradual atherosclerotic process in which plaque deposits have accumulated to narrow the lumen through the blood vessel. Angioplasty and stenting are well accepted amongst interventional physicians, and the long-term outcomes are clinically acceptable. Alternatively, surgery may be employed for those patients who are not suitable for interventional procedures, or for those with a disease state has completely blocked the vascular lumen, leaving it un-crossable by interventional methods. In these cases, the surgical approach provides a physical bypass around the stenosed or occluded vessel, either by an artificial bypass graft, or through the excision and surgical attachment of a vein harvested from another part of the body. However, these two modalities of treatment do not remove the plaque burden in the native vessel, which is a result of the gradual atherosclerotic process. Rather, the action of angioplasty simply applies an outwardly directed radial force to compress the plaque against the vessel wall to expand a small lumen within the stenosed vessel into a larger lumen capable of carrying an adequate supply of blood to the heart tissue under both at-rest and more physiologically demanding conditions, as required by exercise. The introduction of vascular stents affords the physician an additional modality of treatment wherein the stent (a small expandable, tubular metal scaffold) is deployed within the vascular site having undergone angioplasty. The deployed stent maintains the compressed plaque in a compressed state against the vessel wall and maintains the large lumen diameter produced by angioplasty.

The removal of plaque burden is clearly desired from a clinical perspective since it allows the vessel to heal from a more physiological natural base. However, the process of plaque removal has continued to remain a challenge from a device perspective. Ideally, the action of plaque removal should be guided by a visual indicator that the physician may use to distinguish the difference between the plaque itself and the vessel wall. Plaque removal should only be performed up to boundary of the vessel wall, but not include material removal from the vessel wall itself, which may cause a perforation of the vessel wall. The most severve consequence of this type occurs in a coronary artery. The perforation may allow blood to escape fro the blood vessel and into the pericardial sac surrounding the heart. If the perforation remains open, blood may continue to pool in the space between the pericardial sac and the heart, leading to a condition known as haemopericardium. If the process continues, the pooling of blood may become significant enough to compress the heart Itself within the pericardial sac, and prevent the heart from filling with blood, and pumping effectively. This advanced state of haemopericardium is referred to as cardiac tamponade, and requires immediate intervention to seal the perforation in the vessel, and remove the pooled blood within the pericardial sac. Clearly, perforation of the vessel is to be avoided. A like situation may occur within the iliac (peripheral) artery wherein blood may pool into the peritoneal cavity, and if left untreated, will lead to a continuous drop in blood pressure. Immediate intervention to correct the vessel perforation is also required. Interventional (catheter-based) systems have been designed to perform coronary and peripheral atherectomy, but without the advantage of having on-board guidance. A suitable on-board guidance system that may be integrated into an atherectomy catheter, and having the ability to distinguish between plaque and the vessel wall may be either Intravascular Ultrasound as described in U.S. Pat. No. 5,095,911, or Optical Coherence Tomography (OCT) as described in U.S. Pat. No. 5,321,501, 5,459,570, 5,383,467 and 5,439,000. From a development point of view, either ultrasound or OCT may be employed to integrate into the catheter system. However, two significant issues have plagued the development of these types of catheter systems. First, the integration of an atherectomy sub-system-and an on-board ultrasound visualization sub-system within the same catheter may place compromising constraints on the real estate of the catheter system. To date, an integration of these two systems which produces a catheter with a clinically acceptable profile has been prohibitive. Second, in order for an integrated catheter system to be a viable tool for interventional cardiologists and interventional radiologists, the image produced by the on-board guidance system must be correctly interpreted by the physicians. In other words, the images produced by the ultrasound system must be interpreted correctly by the physician so that the physician has the correct information to either continue removing plaque tissue, or to stop the atherectomy procedure because the catheter system has removed all plaque material up to the vessel wall. Unfortunately, this aspect of the procedure can carry a finite degree of risk itself because the images are always under the subjective interpretation of the physician.

Due to the aforementioned degrees of apparatus and user-oriented risk, it would be advantageous for a catheter system to be developed that is simplistic in its design, and that can perform safe atherectomy without the use of subjective on-board visualization. This is the focus of the invention described herein.

### SUMMARY OF THE INVENTION

The catheter system described herein is capable of performing atherectomy along a forward-directed trajectory of the catheters central axis and immediately distal to the catheters atraumatic, rounded distal housing. The catheter's design allows safe advancement through a completely occluded vessel without the use of “on-board” visualization (ultra-sound or Optical Coherence Tomography), and generally uses only fluoroscopic guidance. The catheters first application is to generate a patent pathway through a vascular chronic total occlusion, known as a CTO. From a device or interventional perspective, chronic total occlusions differ from stenosed blood vessels in that catheter systems to treat them cannot be guided over a guide wire through the occlusion since no pathway yet exists through the occlusion. The catheter may however be advanced up to the start of the occlusion over a guide wire. Hence, in this first application the catheter may be advanced over a guide wire through the patent portion of the vessel leading to the occlusion, after which the catheter may only advance through the occluded portion of the vascular occlusion without the use of a guide wire. The second application is to de-bulk a stenosed vessel that is not totally occluded but contains a small but patent pathway that is at least large enough to pass a guide wire there through. In this second application, the catheter may be advanced over a guide wire that has been placed though the stenosed vascular lesion. This is to say that the guide wire passes from the patent portion of the blood vessel from the proximal to the occlusion to the patent portion of the vessel distal to the occlusion. In the second application, the guide wire may remain in its position across the occlusion as the catheter removes vascular plaque as it is advanced over the guide wire. In both applications, this novel technique of atherectomy is referred to as “Forward-Directed” Atherectomy (FDA), and is unique because prior embodiments of atherectomy catheter systems designed to operate within a stenosed blood vessel remove stenotic tissue via a side opening in the catheters distal housing. This forward-directed atherectomy catheter system may be applied to any stenosed mammalian artery or vein.

The first, and most clinically significant application of this catheter system is to forwardly engage total vascular occlusions, and generate a patent pathway from the patent portion of the blood vessel proximal to the occlusion to the patent portion of the blood vessel distal to the occlusion. Occlusions that are at least 3 months in duration and completely block the flow of blood within the vessel are generally known amongst vascular interventionalists as chronic total occlusions (CTOs), and may not be routinely crossable via standard (guide wire) based methods. This new catheter system describes a device and method to generate a patent pathway through the occlusion, thus re-establishing blood flow from the vessel segment proximal to the occlusion to the vessel segment distal to the occlusion. The second application of the catheter system is to de-bulk (remove diseased tissue, or vascular plaque) from stenosed arteries in which the atherosclerotic process has narrowed a segment of the vessel, but a small patent pathway still exists to connect blood flow from the vessel segment proximal to the stenosed region to the vessel segment distal to the stenosed region. In this second application, the catheter system is tracked over a guide wire that has been advanced through the diseased, narrowed segment of the vessel.

The catheter system described herein selectively leverages the differences in material properties of the blood vessel outer wall, which is defined as the tunica adventitia layer, as compared to the properties of the atherosclerotic diseased tissue that lies internal to the tunica adventitia layer. First, the properties of the blood vessel wall will be described, and second, the properties of the atherosclerotic, diseased tissue commonly known as “plaque” will be described.

In a cross sectional view of a healthy artery or vein, (see FIG. 1) the histologically significant layers of the blood vessel wall are identified. The first, inner-most layer of the blood vessel wall is the tunica intima (TI) and is composed of endothelial cells that line the interior surface of the vessel wall, a sub-endothelial layer composed of fibro-elastic tissue, and an outer band called the internal elastic lamina (IEL). The tunica intima defines the boundary of the vessel lumen (L). Moving radially outward, the next layer is the tunica media (TM) and is generally the thickest layer and contains smooth muscle cells amid collagen fibers. The smooth muscle cells are generally arranged in a circumferential or spiral fashion such that they provide support circumferential “tone” to the vessel during the diastolic portion of the cardiac cycle. The next layer is the external elastic lamina (EEL), and is similar to the IEL. The IEL and EEL are thin annular bands that contain the media within. The last, and outermost layer of the blood vessel is the tunica adventitia (TA), and is composed mainly of collagen and elastic fibers. The adventitia is the outer, elastic but absolute boundary to natural or imposed radial expansions of the vessel. For the purposes of the discussion contained herein, the tunica adventitia will be defined as the outer boundary of the blood vessel wall.

The diseased, stenosed tissue on the other hand has no particular structure other than being predictably random in its construction. See FIG. 2. However, the major constituents of plaque may be generally categorized as a random mix of thrombus deposits (T), fibro-calcific deposits (FC), and discrete calcium deposits (DC). The deposition of plaque within the vessel may substantially reduce the cross sectional area of the lumen (L) as shown in FIG. 2. It is well documented through historical clinical evidence that the action of angioplasty, which places the vascular plaque under pressure between the adventitial layer and the balloon catheter, compresses the plaque to increase the lumen diameter at the site of the previously stenosed, diseased pathway in the vessel. The consistency of the plaque may actually vary quite substantially from vessel to vessel and patient to patient. In one extreme, the disease state may be highly calcified, but as part of a fibro-calcific matrix. In the other extreme, the plaque may consist of a higher concentration of thrombus and fibrotic tissue. In either case and most importantly, the overall properties of typical plaque would be considered to have less of an elastic component than that of the tunica adventitia. The greater degree of elasticity afforded by the tunica adventitia, as compared to the random and relatively non-elastic plaque is the basis of design for this invention.

Returning now to the elastic properties of the adventitia, natural expansion and contraction of a healthy vessel occurs during the normal systole-diastole cycle (normally 120 mm Hg to 80 mm Hg), wherein the diameter of the blood vessel will expand slightly during systole, and return to its “at-rest” state during diastole. In a healthy vessel, the degree of expansion is a composite of the elastic properties of the adventitia, IEL and EEL, the media and the myocardial tissue that surrounds the vessel. What is important to note is that in a healthy vessel, and during a normal cardiac cycle, the “limit of expansion” of the adventitial layer is not tested. In other words, the adventitia may not reach the limit of its elastic expansion when imposed upon it by normal systolic blood pressure forces. However, the condition of “testing the expansion boundaries” of the adventitia is only encountered in a diseased vessel, when an external force is applied to the blood vessel, namely that of percutaneous transluminal coronary angioplasty, or PTCA. In a diseased blood vessel, the inner layers of the vessel may be destroyed, namely the intima, IEL, media, and the EEL. The degree of destruction of these layers may not be ascertained by fluoroscopy, and can only be ascertained via a microscopic histological observation of the excised vessel. During PTCA, the blood vessel may undergo tremendous stress wherein the adventitial boundary counters the force applied from the balloon catheter, which may range from 6 atmospheres to 20 atmospheres, and the layer of plaque between the two is under compression. It is critical to note that during this process a point of radial stress will be reached wherein the adventitia may no longer act in an elastic mode, and the expansion of the vessel will approach an asymptotic limit with the adventitia ultimately acting as a restrictive circumferential boundary. Without this physical boundary provided by the tunica adventitia layer, the balloon catheter would have no support onto which apply its force against the plaque.

The foregoing discussion identifies pertinent physical characteristics of the tunica adventitia upon which this new catheter system leverages in its design, and allows this novel catheter system described herein to operate in the two applications previously described, namely via the action of forward-directed atherectomy, to establish a patent pathway through a chronically occluded or stenosed blood vessel. Having now described the differences between the physical properties of the tunica adventitia layer and vascular plaque upon which this catheter design is derived, the main design features of the catheter system will now be described.

The invention described herein consists of a flexible catheter system usable to remove vascular plaque within a blood vessel. Six design attributes define the pertinent aspects of the invention: 1) a convex shaped, atraumatic distal housing affixed to the catheter shaft, with a pattern of openings, or cells to communicate with the interior of the housing, the “scaffolding” or struts between the openings maintaining intimate forward-directed contact with the vascular plaque but allowing the plaque tissue to slightly impinge into the openings, and into the interior of the catheter, 2) an internal rotational cutter having a) at least one cutting blade, the cutting edges designed to translate along the interior surface and contour of the distal housing struts or directly against the interior surface of the struts so as to shave the plaque material that has impinged through the distal housing openings and into the interior space within the distal housing, and b) a central lumen to allow the passage of a guide wire or fluids, or both simultaneously, 3) a flexible, torqueable catheter shaft, the distal annular end of which is connected to the proximal annular end of the distal housing, 4) an internal, flexible and torqueable drive shaft connected to, and capable of delivering rotational torque to the internal rotational cutter, the drive shaft having a central lumen allowing the passage of a guide wire or fluids, or both simultaneously, 5) a port positioned at the proximal portion of the catheter and in communication with the annular lumen between the inner surface of the catheter shaft and the outer surface of the rotating, flexible drive shaft, and attachable to an external vacuum source, wherein plaque material that has been shaved off by the internal cutter may be evacuated from the annular space and removed from the catheter through the port, and 6) a drive unit connected to the proximal end of the rotatable, flexible drive shaft for delivering rotational motion to the drive shaft and internal rotational cutter. An optional design feature attached to the internal rotational cutter is a cylindrical fluid-propelling component composed of an external cylindrical housing or ring, a central hub containing a lumen capable of passing a guide wire or the passage of fluids or both simultaneously, and pitched fins connected there between. In a preferred embodiment, the fluid-propelling component may reside between, and be connected to the internal rotational cutter on one end, and the flexible, torqueable drive shaft on the other end. Hence the drive shaft, the fluid-propelling component and the internal cutter may be rotated as a unified system. In one rotational direction of the fluid-propelling component, fluid within the distal housing will be propelled in a proximal direction. Alternatively, if the rotation of the fluid-propelling component is reversed, fluid in the distal housing will be propelled in the opposite direction. The main purpose of the fluid-propelling component is to assist in removing shaved plaque from the interior of the distal housing, and translate it into the annular space between the drive shaft and the catheter shaft, wherein the vacuum system may continue in translating the plaque particles proximally through the catheter, to be removed via the proximal port.

Forward advancement of the catheter within a stenosed blood vessel, and depending upon the vessels curvature, may be accomplished with or without tracking the catheter over a guide wire. If desired by the interventionalist, tracking the catheter without a guide wire in the vessel may be performed safely since the distal housing of the catheter is rounded and relatively large with respect to the size of the vessel, and the distal catheter shaft is designed with great flexibility. As an example of comparison of distal housing diameter and blood vessel lumen of a coronary artery, a nominal diameter of the distal housing may be 0.100″-0.120 (˜2.5 mm˜3.0 mm) and the native (non-diseased) portion of the vessel proximal to the occluded or stenosed area may be 0.140″ (˜3.5 mm). Upon engagement of the distal housing against the vascular plaque, the plaque may impinge through the openings of the distal housing, and into the interior of the distal housing, the depth of impingement being dictated by the composition of the plaque, the area of the openings, and the wall thickness of the distal housing. As an example, plaque composed of thrombus and fibrous growth will exhibit more pliability and impinge into the interior of the housing to a greater degree than plaque composed of localized calcium deposits and fibrous growth. In general, thrombus and fibrous growth will have more of a visco-elastic property, whereas a non-homogeneous mix of localized calcium deposits and fibrous growth will display reduced visco-elastic properties. However, even a localized rigid calcium deposit, which may typically exhibit an irregular surface contour, may slightly impinge through an opening of the distal housing and into the interior of the distal housing by virtue of the “curvature ” of the opening itself, i.e. the opening is produced through the thin wall structure of the convex, rounded distal housing. Plaque that has impinged through the distal housing openings and into the interior of the distal housing is subsequently engaged by the blade edges of the rotational cutting element. This simple, incremental action will slice or shave a small portion of the plaque that has impinged through any of the multiple openings and into the interior of the distal housing. If the catheter remains in this initial orientation at the stenosed or occluded site in the vessel, the tissue within the openings will the shaved off within the interior of the distal housing, and the tissue in immediate contact with the distal housing struts will not have the opportunity to impinge through the distal housing openings. However, if the catheter shaft and distal housing are now rotated, the portion of the plaque tissue that was previously in immediate contact with the distal housing struts will now have the opportunity to also impinge through the openings and into the interior of the distal housing. As this process continues wherein the catheter shaft and distal housing are rotated while the distal housing remains in intimate contact with the vascular plaque, the mass of plaque tissue immediately distal to the catheter distal housing openings will be incrementally shaved off within the distal housing and loaded into the annular space between the internal drive shaft and the catheter shaft. However, in order to efficiently evacuate the particles of shaved plaque within the distal housing and catheter shaft, the particles may be placed in a fluid suspension by infusing saline within the lumen of the rotating, flexible drive shaft to exit at the distal portion of the catheter. Further, vacuum may be applied via the catheter proximal port and within the space between the flexible drive shaft and the catheter shaft, and the shaved portions of vascular plaque, now in a saline suspension, may be evacuated proximally through the catheter via this annular space. In addition, the optional fluid-propelling component may serve to continuously remove the shaved plaque material from the immediate vicinity of the pattern of openings within the distal housing, allowing the openings to continuously receive the subsequent impingement of vascular plaque. This process may continue wherein the vascular plaque is incrementally shaved within the interior of the distal housing, and evacuated through the annular space between the rotating drive shaft and the catheter shaft. As vascular plaque tissue is incrementally removed, the distal housing of the catheter shaft may be rotated and advanced forward through the chronic total occlusion or stenosed blood vessel.

In a first preferred embodiment, the exterior contour of the distal housing has been described as having openings that allow tissue to enter the openings along a proximally directed vector that is parallel to the catheters central axis. In this first preferred embodiment the cells do not extend into the cylindrical, lateral surface of the housing.

In the aforementioned description of the catheter system's advancement through the vascular plaque, it has been assumed for reasons of simplicity that the distal catheter housing remains in exclusive contact within the vascular plaque. However, in practice this scenario is theoretical at best, and due to normal vascular tortuosity (curvature), the outer surface of the catheter distal housing will, at some point come into contact with the vessel wall itself. This is the expected and normal course in which the catheter will translate within the bounds of the vessel wall. As described earlier, in many cases all layers of the vessel wall may not be present in a stenosed or occluded blood vessel. However, at a minimum, the adventitial layer will be present. Under this scenario, three critical factors require explanation to validate the catheters ability to safely navigate through an occluded or stenosed blood vessel. These factors are: 1) the physical properties differentiating the. adventitial wall of the vessel from those of the plaque, 2) the shape and dimensions of each of the distal housing openings, or cells, and 3) the thickness of the distal housing itself. The first of the three factors was previously described. The second and third factors, the shape and dimension of each distal housing cell, and the thickness of the distal housing, respectively, are interrelated and are designed to allow plaque tissue to enter through the openings in the distal housing, yet not allow or limit the tunica adventitia layer of the blood vessel from entering into the interior of the distal housing. First, the shape of each distal housing cell and the wall thickness of the distal housing into which each cell is produced are designed to allow the impingement, or ingress of vascular plaque which is typically visco-elastic, and non-homogeneous in its make-up, through the cells and into the interior of the distal housing. Second, the interrelationship between the cell dimensions and the wall thickness of the distal housing are chosen to not allow or limit the impingement or ingress of the vessel wall (tunica adventitia) into the interior of the distal housing. Preventing or limiting the impingement of the tunica adventitia into the interior of the housing is the more important of the two issues, since this relates directly to the safety of the catheter system.

As previously described, the adventitia exhibits elastic type properties up to a point of maximum strain. Strain is defined as the degree of stretch or elongation of a material that is undergoing stress (force input). The elastic properties of the tunica adventitia allows the vessel wall to “stretch” over the outer surface of the distal housing, and more specifically allows the tunica adventitia to stretch across the struts of each open cell. As the tunica adventitia is stretched across the struts of each cell, the tunica adventitia becomes taught, and under ideal conditions the tunica adventitia will not be able to impinge or ingress into the cell or into the interior of the distal housing. However, in practice slight impingement of the tunica adventitia though the cells and inward past the imaginary boundary of the inner surface of the distal housing may occur. However, preventing or limiting the tunica adventitia from entering the interior of the distal housing (translating radially inward past the inner surface of the distal housing) is accomplished by adjusting the size, configuration and thickness of the distal housing. In a preferred embodiment, FIG. 5b shows one of the cells from a tangential perspective. This view demonstrates the “straight-line” pathway the tunica adventitia will assume when stretched over Strut A and Strut B. FIG. 5b demonstrates that if the tunica adventitia was stretched across Strut A and Strut B, it would not be able to enter into the interior of the distal housing. However, as mentioned earlier, at first the inner surface layer of the wall of the tunica adventitia may enter just inside the inner surface of the distal housing. However, as increased engagement force is used to advance the catheter into the vascular plaque, the tunica adventitia will experience even greater relative stretch force over the struts, which will serve to tighten the tunica adventitia over each cell, thereby making its potential ingress into the cell less likely.

It may seem that placing the vessel wall's tunica adventitia under stress may not be desirable, since maintaining the integrity of the vessel wall is one of the most important clinical consideration in any interventional vascular procedure. However, returning again to the operational basis of percutaneous transluminal coronary angioplasty or PTCA, this method has been utilized successfully for 30 years. It's success is only possible due to the tenacious, elastic properties of the adventitia. This new invention leverages these same properties to perform the action of Forward-Directed Atherectomy.

### BRIEF DESCRIPTIONS OF THE DRAWINGS

FIG. 1: Cross section of a normal, healthy blood vessel.

FIG. 2: Cross section of a diseased vessel, containing plaque.

FIG. 3a: Isometric view of a first preferred embodiment of the catheter system.

FIG. 3b: Isometric view of the distal housing of a first preferred embodiment of the catheter system.

FIG. 3c: Isometric view of the internal rotational cutter of a first preferred embodiment of the catheter system.

FIG. 3d: Cross section view of a first preferred embodiment of the catheter system.

FIG. 4a: Isometric view of a second preferred embodiment of the catheter system.

FIG. 4b: Isometric view of the distal housing of a second preferred embodiment of the catheter system.

FIG. 4c: Isometric view of the internal rotational cutter of a second preferred embodiment of the catheter system.

FIG. 4d: Cross section view of a second preferred embodiment of the catheter system.

FIG. 5a: Side view of the catheter distal housing showing the relationship between the convex surface and the cylindrical or lateral surface.

FIG. 5b: Tangential view of the outer surface of the distal housing.

FIG. 6: Catheter shaft.

FIG. 7: Isometric view of the optional fluid-propelling feature and the drive internal shaft.

FIG. 8: Isometric, cut-away view of the fluid-propelling feature, the rotational cutter and the drive shaft.

FIG. 9: Isometric, cut-away view of a preferred catheter embodiment including the fluid-propelling feature.

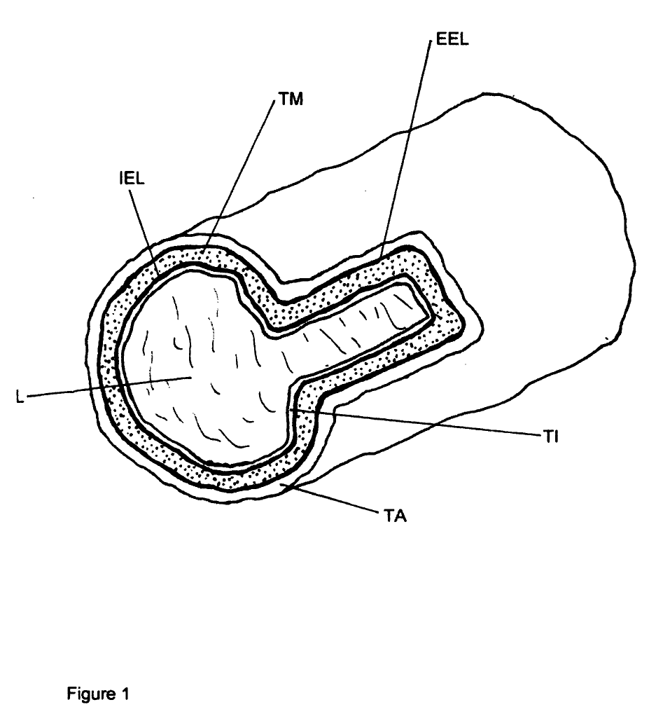

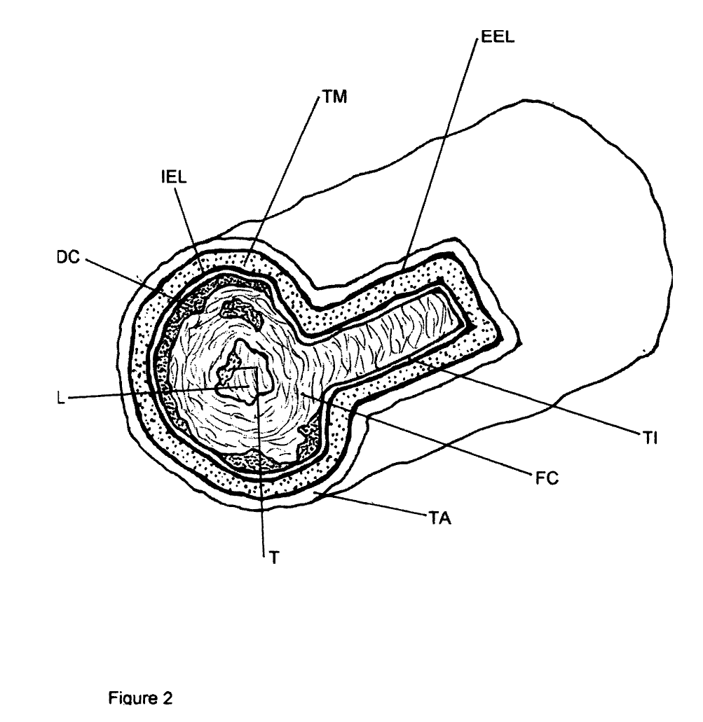

### DETAILED DESCRIPTION

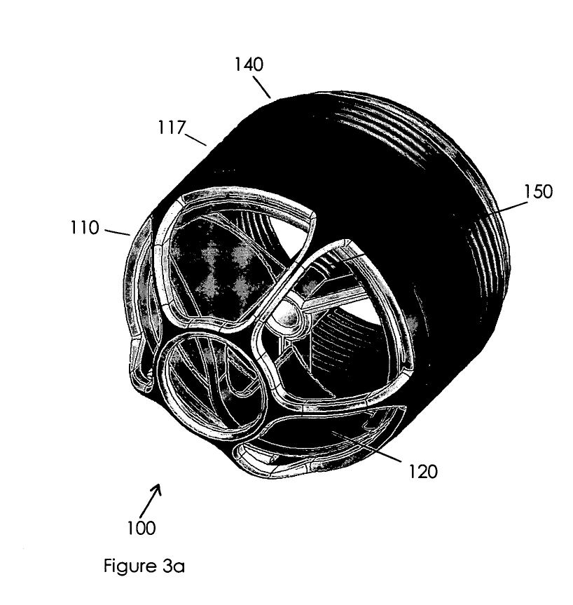

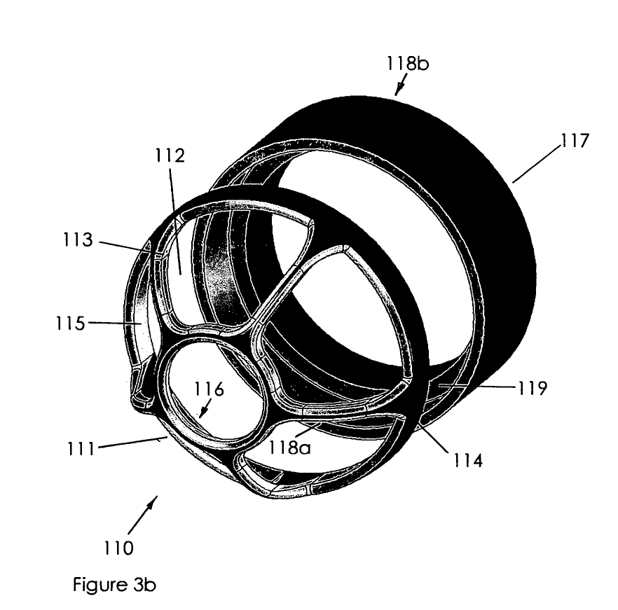

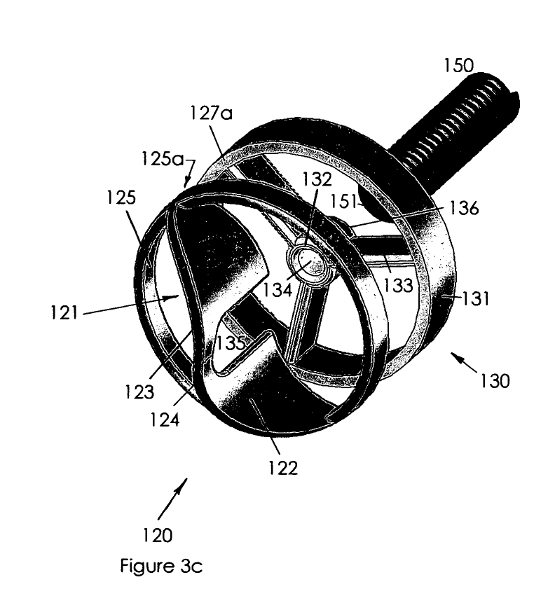

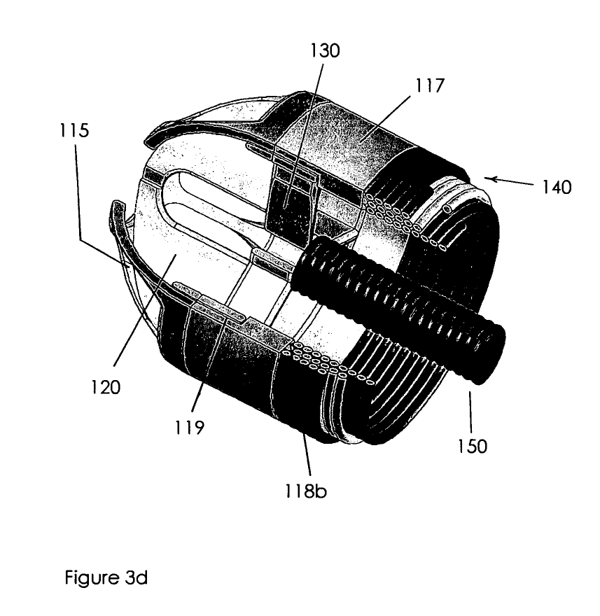

A forward-directed atherectomy catheter is described for generating patent pathways through vascular total occlusions and de-bulking stenosed blood vessels. A first preferred embodiment of the catheter system 100 is shown in FIG. 3a. The main components are the distal housing assembly 110, the internal rotational cutter assembly 120, the torqueable, flexible catheter shaft assembly 140, and the torqueable, flexible internal drive shaft 150.

A first preferred embodiment of the distal housing assembly is shown in FIG. 3b. The distal housing assembly is comprised of two separate components for ease of manufacturing. The distal portion is the convex housing 111 and the proximal portion is the collar housing 117. The convex housing contains multiple openings or cells 112 separated by struts 113, an outer surface 114, an inner surface 115, and a proximal annular face 116. The edges of each strut are rounded and are part of the outer surface 114 so as to present an atraumatic surface when the struts are in contact with the vessel wall. The collar housing 117 has a distal annular face 118a, a proximal annular face 118b, and an inner annular recess 119. The proximal annular face 116 of the distal housing is attached to the distal annular face 118a of the collar housing 117, and the proximal annular face 118b of the collar housing 117 is attached to the distal end of the catheter shaft.

Both the convex housing 111 and the collar housing 117 may be fabricated from any number of machineable materials, but the preferred choice is that of stainless steel or non-iron containing compounds primarily composed of nickel, chromium and cobalt such as MP35N supplied by Fort Wayne Metals. These materials are chosen for their strength and their ability to be machined via Swiss Screw Machine methods, and Fine Wire Electric Discharge Machining or “EDM”, both of which are preferred methods to fabricate the distal housing. The convex shape of the housing is best machined using Swiss Screw Machining, which is a metal turning method fundamentally similar to that of a metal-working lathe, however the Swiss Screw Machine is able to hold much tighter tolerances, and is the proper choice for machining small components with radial symmetry. As well, the collar housing may be machined using Swiss Screw Machining. The cells in the distal housing may also be machined using a Swiss Screw machine, however Electric Discharge Machining may lend itself as a preferred machining method. In Electric Discharge Machining, the component is electrically connected to a high voltage circuit and submerged in a conductive water-based solution. An electrified small wire, typically on the order of 0.002″ to 0.010″ diameter is connected to the other end of the circuit. The electrified wire is programmed to translate a pathway through the component in the shape of the feature to be machined. As the electrified wire makes contact with the component, the high voltage circuit is completed via the conductive water-based solution, and microscopic ablation of the metal occurs as the wire translates along the programmed pathway through the part. EDM can produce exceedingly accurate tolerances to within tens of thousandths of an inch, and is used in countless applications to machine small parts that are along the same size, shape and complexity as the distal housing.

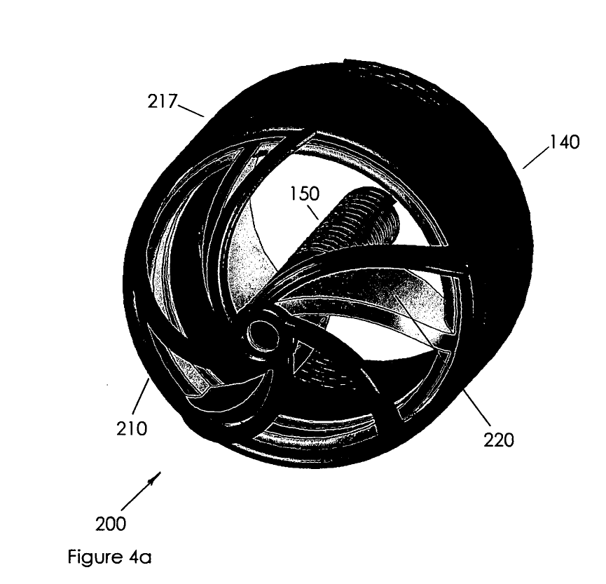

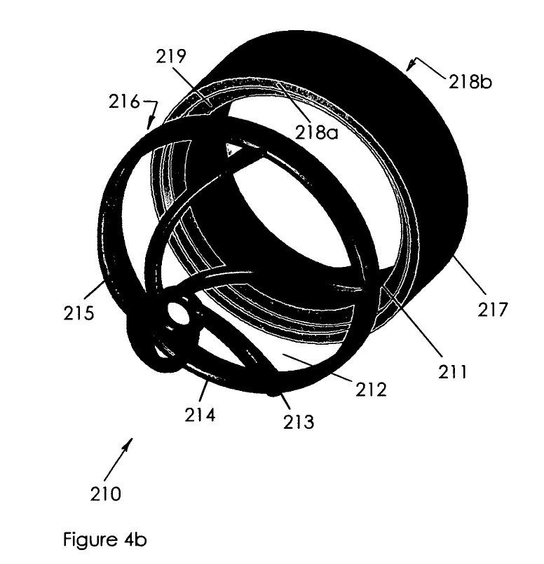

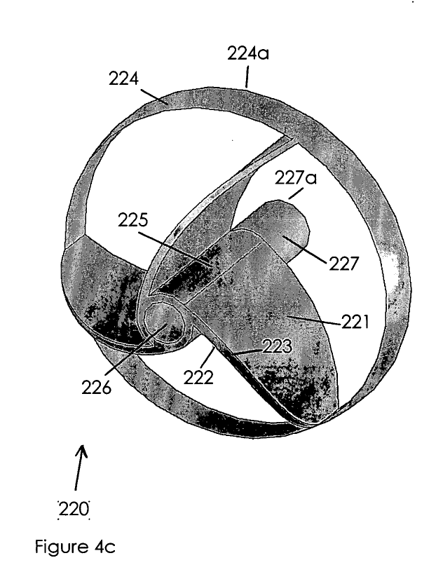

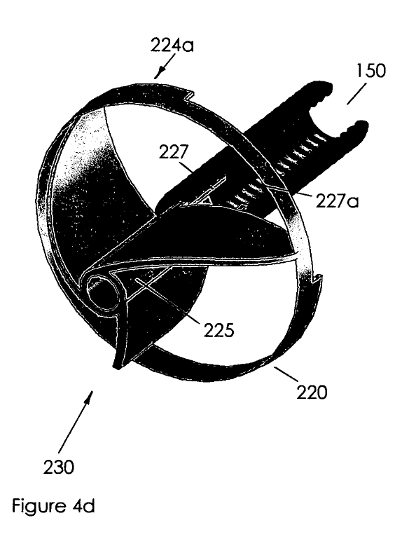

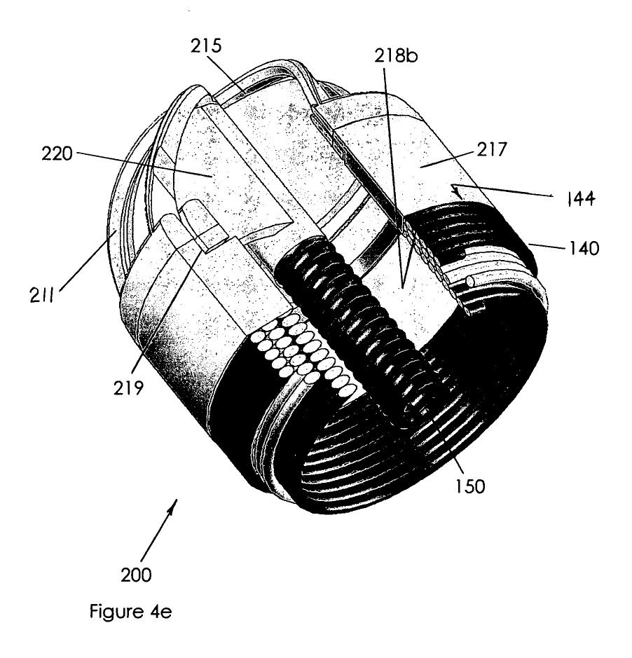

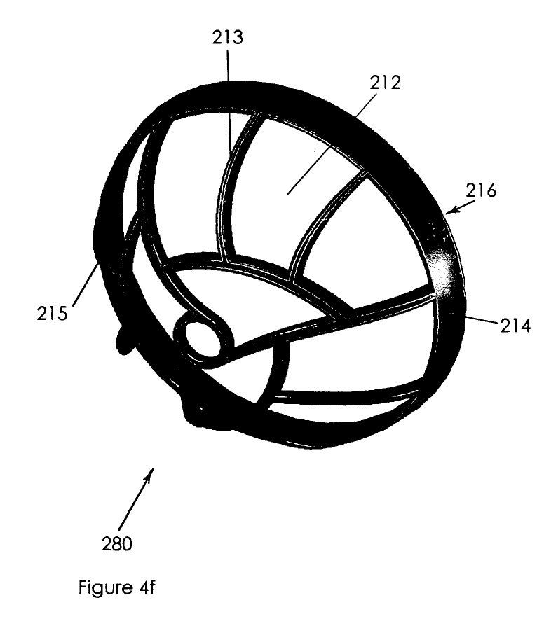

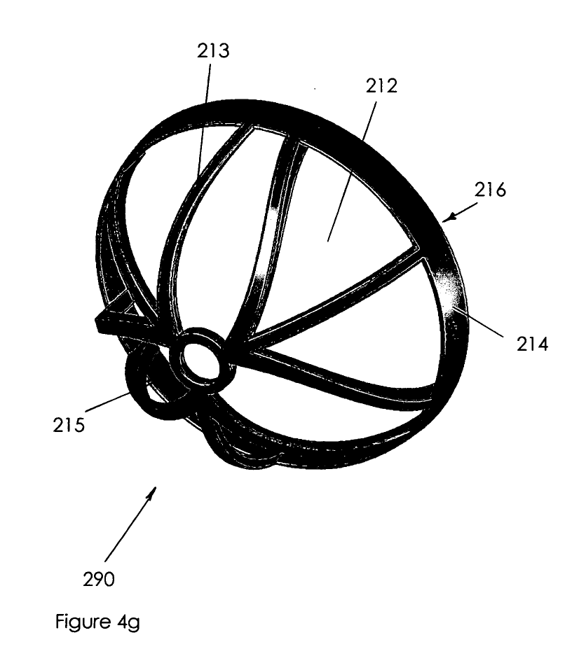

The first preferred embodiment of the distal housing assembly shown in FIG. 3b utilizes five cells, but the number of cells is not so restricted. The second preferred embodiment shown in FIG. 4b utilizes five cells of a different configuration, but again the number of cells is not so restricted. A lesser or greater number of cells may also serve equally beneficial so as the shape and size of the cells, along with the thickness of the distal housing are adjusted to as to prevent or greatly limit the ingress of tunica adventitia tissue through the cells.

The distal convex housing 111 and the collar housing 117 may be fabricated of similar metals, and the preferred method of joining the components to each other is by laser welding. In this process, a fine laser beam is directed at the interface between the two components. Only the localized area of each component exposed to the beam is heated to a point of melting, wherein the components are fused at that location. The beam is then translated along the interface pathway between the components to complete the weld process. In general, the diameter of the distal housing may vary according to the vessel diameter in which it is used in the body. In the coronary arteries, the distal housing diameter may vary from 0.080″ to 0.120″, and in the peripheral arteries, the diameter may range from 0.100″ to 0.200″ in diameter, but the catheter system is not limited by these dimensions.

A first preferred embodiment of the internal rotational cutter assembly 120 is shown in FIG. 3c. In this preferred embodiment, the internal cutter 121 may contain at least one cutting blade 122, but not limited by this number, with two cutting edges 123 and 124. At least one section of the radial exterior end of the blade is connected to an annular ring 125. Attached to the internal cutter 121 is the torque drive 130. The torque drive consists of an annular ring 131, a central hub 132, and connection fins 133. The central hub 132 also contains a central lumen 134 that may be used for the passage of a guide wire, or fluids, or both simultaneously. A central cutout 135 in the blade allows a guide wire to be advanced from the lumen 134 and into the cutout 135 whereby it may then be advanced distally through the central opening of the distal housing. The distal face 151 of the internal drive shaft 150 connects via laser welding to the proximal face 136 of the central hub 132. In one method, the distal face 127a of the annular ring mates to the proximal face 125a off the internal rotational cutter's annular ring 125. The internal rotational cutter may be fabricated of like materials, and utilize the same manufacturing methods as those described for the convex housing and the collar housing. Alternatively, even more robust materials may be used in the construction of the internal cutter. These materials may include ceramic formulations, or carbide type materials. Both of these material types may offer increased ability to maintain the cutting edge of each blade during use. In a preferred embodiment, the internal rotational cutter is designed such that the cutting leaves may be the same number as that of the repeating cell pattern in the convex housing, and the radial cutting edges of the internal cutter may either be in contact with the interior surface of the convex housing, or in close proximity to the interior surface of the convex housing. In either embodiment, as the leaves of the internal rotational cutter are rotated within the distal housing, plaque material that has impinged to within the interior of the housing is caught in between the cutters cutting edge, and the convex housing strut(s) between the open cells of the distal housing. The plaque material is subsequently shaved off within the interior of the distal housing. Concurrent with this action, if saline solution is infused through the central lumen of the catheter, and vacuum is applied to the catheters proximal port and within the space between the catheter shaft and the drive shaft, the suspension of plaque particles within the saline solution within the distal housing may be evacuated from the interior of the distal housing and into the main body of the catheter system, allowing subsequent plaque to be shaved from the vessel.

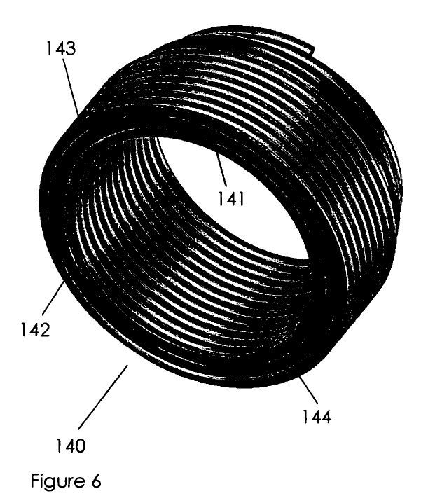

FIG. 3d shows a cross section of the distal portion of the catheter system. The torque drive 130 of the internal rotational cutter assembly 120 is shown nested within the recessed annular cutout 119 of the collar housing 117. The dimensions of these components may be held to very tight tolerances, and thus the internal rotational cutter may be accurately nested between the interior surface 115 of the convex housing and the recessed annular cutout 119 of the collar housing 117. The nesting of the internal rotational cutter within the distal housing also prevents the internal rotational cutter from changing its position within the distal housing. Connected to the proximal face 118b of the collar housing is the distal face 144 of a preferred embodiment of a catheter shaft assembly 140 as shown in FIG. 6. A standard catheter shaft assembly may utilize numerously practiced fabrication methods by those fluent in the art, namely variations of a polymer laminated, braided stainless steel wire mesh configuration. There are numerous lay-ups using this configuration, however the preferred shaft embodiment consists of a construction commonly referred to as “Tri-Plex” as shown in FIG. 3a, FIG. 4a and FIG. 6. Triplex consists of a layered configuration of three separate coils. The wire used to make the coils may be stainless steel or the nickel, chromium and cobalt alloys previously described. The outer coil and inner coil are wound in one direction, for instance clockwise, and the middle coil is wound counter-clockwise. Normally, individually wound coils have no substantial means to prevent their stretching. However, when wound as part of a counter-wound lay-up, the individual coils interlock with each other to control shaft stretching to an acceptable minimum, as well as to afford outstanding flexibility and torque transmission in both the clockwise and counter-clockwise directions. Further, from a manufacturing perspective, the distal end of each of the coils may be welded to each other, producing a circumferential weld ring that may subsequently be welded to the proximal ring of the collar housing. Collectively, this produces an extremely flexible, bi-directionally torqueable shaft assembly that is weldable to the distal housing, producing a unified catheter construction. Further, the Tri-Plex catheter shaft may be directly coated with a hydrophilic coating, such as that supplied by Surmodics Corporation. The coating provides a hydrophilic film on the surface of the Tri-Plex catheter shaft and may greatly aid its guidance and translation through a totally occluded, or heavily stenosed vessel. Alternatively, the outer surface of the catheter shaft may be laminated with a relatively low durometer polymer, such as 35D Pebax or Pellathane to produce a smooth surface, as opposed to the very small surface features of the outer coil. The polymer laminate may also be coated with the hydrophilic coating to produce a lubricious outer surface. As well, the inner surface of the shaft assembly may also employ a polymer laminate. In this case, a fluoropolymer such as polyteterafluorethylene (PTFE) may line the inner diameter surface of the inner coil. This polymer may serve to lessen the rotational friction of the drive shaft against the inner surface of the catheter shaft. The diameter of the catheter shaft may be of the same diameter as the distal housing, or it may be smaller in diameter to facilitate its translation through the passage provided by the atherectomy of vascular plaque tissue. In general, the diameter of the shaft may vary according to the vessel diameter in which it is used in the body. In the coronary arteries, the shaft outer diameter may vary from 0.080″ to 0.120″, and in the peripheral arteries, the diameter may range from 0.100″ to 0.200″ in diameter, but the diameter is not necessarily limited to these ranges. The wire diameter used to wind the coils may range from 0.004″ to 0.010″, but is not necessarily limited by this range. An alternative coil construction may be fabricated with a flat wire such as 0.004″×0.012″. Using the aforementioned wires to construct the coils, the composite Tri-Plex may have a minimum wall thickness of approximately 0.012″. Using the same preferred embodiment, if the outer diameter of the Tri-Plex is 0.120″ (3 mm), the inner diameter of the catheter shaft will be 0.096″ (2.4 mm).

FIG. 4a shows a second preferred embodiment 200 of the catheter system. The main components consist of the distal housing assembly 210, the internal rotational cutter 220, the torqueable, flexible catheter shaft assembly 140, and the torqueable, flexible internal drive shaft 150.

A second preferred embodiment of the distal housing assembly 210 is shown in FIG. 4b. In the preferred embodiment shown in FIG. 4b, the distal housing assembly is comprised of two separate components for ease of manufacturing. The distal portion is the convex housing 211 and the proximal portion is the collar housing 217. The convex housing contains multiple openings or cells 212 separated by struts 213, an outer surface 214, an inner surface 215, and a proximal annular face 216. The edges of each strut are rounded so as to present an atraumatic surface when the struts are in contact with the vessel wall. Alternate embodiments 280 and 29 of the convex housing are shown in FIG. 4f and FIG. 4g respectively. The collar housing 217 has a distal annular face 218a, a proximal annular face 218b, and an inner annular recess 219. The proximal annular face 216 of the distal housing is attached to the distal annular face 218a of the collar housing 217. As with the first preferred embodiment, both the convex housing 211 and the collar housing 217 may be fabricated from any number of machineable materials, but the preferred choice is that of stainless steel or non-iron containing compounds primarily composed of nickel, chromium and cobalt such as MP35N supplied by Fort Wayne Metals.

A second preferred embodiment of the internal rotational cutter 220 is shown in FIG. 4c. In this second preferred embodiment, the internal rotational cutter 220 contains three cutting blades 221 but is not limited by this number. The cutting blades have at least one cutting edge 222 that is active as the internal rotational cutter is rotated in one direction, and another other cutting edge 223 that is active when the internal rotational cutter is rotated in the opposite direction. At least one section of the radial exterior end of each blade is connected to an annular ring 224 having a proximal face 224a, and at least one section of the radial interior end of each blade is connected to a central hub 225. The central hub 225 also contains a proximal face 227a, and a central lumen 226 that may be used for the passage of a guide wire, or fluids, or both simultaneously. In one embodiment, a proximal extension 227 of the central hub may be inserted into the distal end of the internal drive shaft 150 as shown in the rotational drive/internal rotational cutter assembly 230 in FIG. 4d, or alternatively the drive shaft 150 may be aligned as a butt joint and laser welded to the proximal face 227a of the proximal extension 227. FIG. 4d shows a sectional view of an internal cutter/drive assembly 230 of the internal rotational cutter 220 attached to the drive shaft 150 via the insertion method.

FIG. 4e shows a cross section of the distal portion of the second preferred embodiment of the catheter system 200. The internal rotational cutter 220 is shown nested between the inner annular recess 219 of the collar housing 217 and the inner surface 215 of the distal housing 211. The dimensions of these components may be held to very tight tolerances, and thus the internal rotational cutter may be accurately nested between these surfaces. The nesting of the internal rotational cutter within the distal housing also prevents the internal rotational cutter from changing its position within the distal housing while under rotation. Connected to the proximal face 218b of the collar housing is the distal annular face 144 of a preferred embodiment of a catheter shaft assembly 140 as shown in FIG. 6.

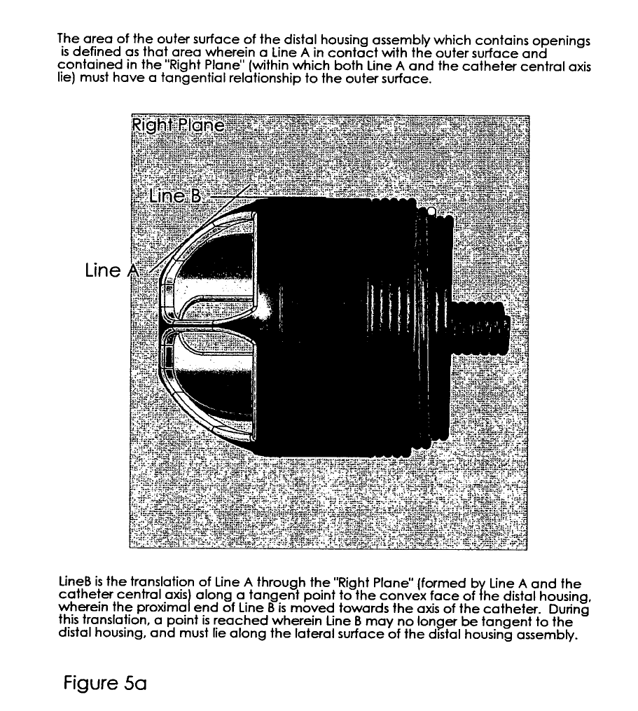

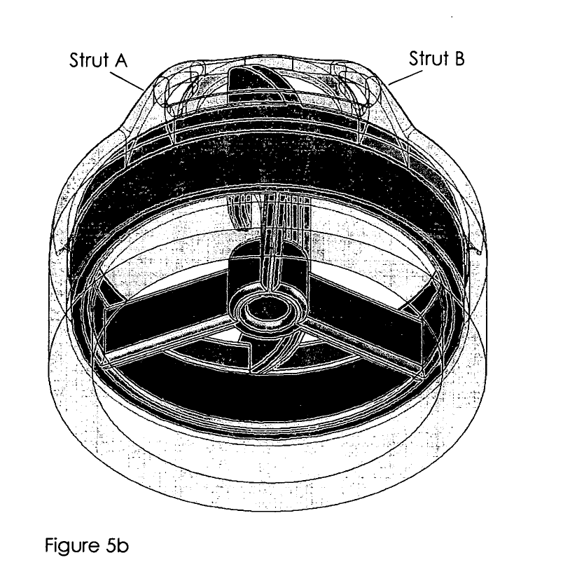

Referring to FIG. 5a, the outer surface of the housing that contains the openings is defined as any portion wherein a Line A lies tangential to the surface, and also lies in a plane produced by Line A and the catheters central axis. Via this definition, Line A may never lie parallel with the catheter's central axis. Wherever Line A may be translated within this plane, and still remain tangent to the surface of the distal housing, the outer surface containing the openings is defined. In contrast, a lateral cylindrical portion of the housing's surface may be present, and is defined as that region wherein a line drawn in the same plane and in contact with the housing's surface may not have a tangential relationship to the housing, rather the line lies along the surface of the lateral, cylindrical portion of the distal housing and be parallel with the central axis of the catheter system. This line is shown as Line B in FIG. 5a. The position and orientation of the cells in the distal housing dictate that the plaque material enters the cells along a vector that is parallel to the catheters central axis, and define the catheter system as performing forward-directed atherectomy. Plaque material enters the openings along this axis and is removed from the volume of space immediately distal to the catheter's distal housing. Upon rotation of the catheter shaft and the repeating of impingement of plaque material through the cells in the distal housing and subsequent shaving process, the catheter is allowed to move forward into the stenosed or occluded vessel, via the space created by the removal of plaque.

FIG. 6 shows a preferred embodiment of the Tri-Plex catheter shaft system 140, consisting of the inner coil 141, the middle coil 142, the outer coil 143, and the distal face 144. The inner coil and outer coil are wound in one direction, for instance clockwise and the middle coil is wound in the opposite direction, in this example counterclockwise. The surface features of the filers (individual turns of a coil) may overlap and inter-digitate in such a manner that the shaft assembly becomes unified, affording it excellent torque control and flexibility combined with a low degree of shaft stretch. The materials used to construct the shaft assembly may be of stainless steel or non-iron containing compounds primarily composed of nickel, chromium and cobalt such as MP35N supplied by Fort Wayne Metals. These materials lend themselves to laser welding of the shaft assembly to the collar housing, which may be fabricated of similar materials.

A preferred embodiment of the internal drive shaft 150 is shown in FIG. 4a and FIG. 4d. In this embodiment, the drive shaft is constructed of a single wound coil. The direction of the winding may be in either direction, but a preferred method may be to wind the coil clockwise (as looking proximal to distal along the drive shaft axis) if the internal cutter is to be rotated in a counter-clockwise direction. In this way, the rotational force upon the drive shaft will serve to “tighten” the coil in its wound configuration. In a second embodiment, the drive shaft may be fabricated from a layered coil configuration similar to that described for the catheter shaft, however instead of three layered coils only two are used. This configuration is referred to as a “Bi-Plex” and is utilized for greater flexibility, however torque transmission is afforded generally in one rotational direction only. If the direction of the internal cutter is again rotated in a counter-clockwise direction, then the outer coil is wound in a clockwise direction, and the inner coil is wound in a counter-clockwise direction. This allows the outer coil to “squeeze” onto the inner coil, thus unifying the interlock between the inner and outer coils and allowing transmission of torque in a counter-clockwise manner. However, the internal drive shaft may not be limited to a Bi-Plex coil assembly, and may also utilize a Tri-Plex, or a braided, laminated shaft construction as previously described. The wires used to wind the coils of the drive shaft may be comprised of stainless steel, or nickel-chromium-cobalt type alloys, but are not so limited. As shown in FIG. 4d, the proximal extension of the internal cutters central hub 225 is connected to the distal segment of the drive shaft via an overlapping type of joint. An end-to-end joint may also be configured, but in either case, in a preferred embodiment using like materials the drive shaft and the internal cutter may be joined via laser welding as described previously. The minimum inner diameter and outer diameter dimensions of the internal drive shaft are dictated by the functional requirements of its internal lumen, which is designed to pass a guide wire, or fluids, or both simultaneously. As an example, if this lumen is designed to pass a standard 0.014″ coronary guide wire, the internal diameter of the drive shaft may be 0.016″. If the drive shaft is wound from a single 0.008″ diameter wire, the outer diameter of the drive shaft will be 0.032″. Alternatively, the drive shaft may be fabricated of a Bi-Plex type of shaft, using 0.004″×0.012″ wire. This configuration may be desirable in some applications since the Bi-Plex would afford a greater degree of flexibility yet still deliver adequate torque transmission.

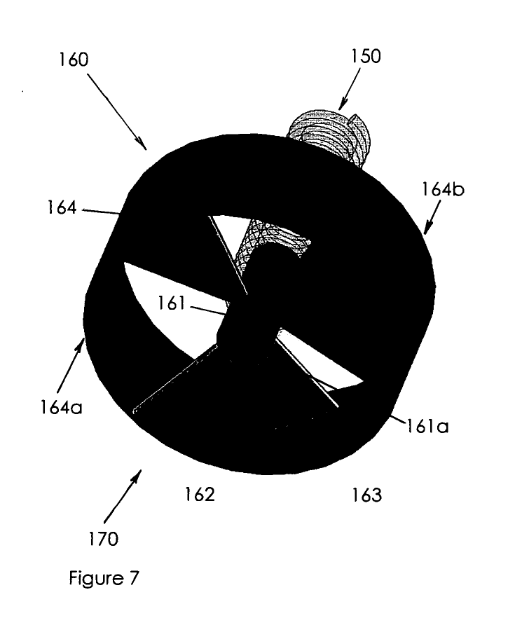

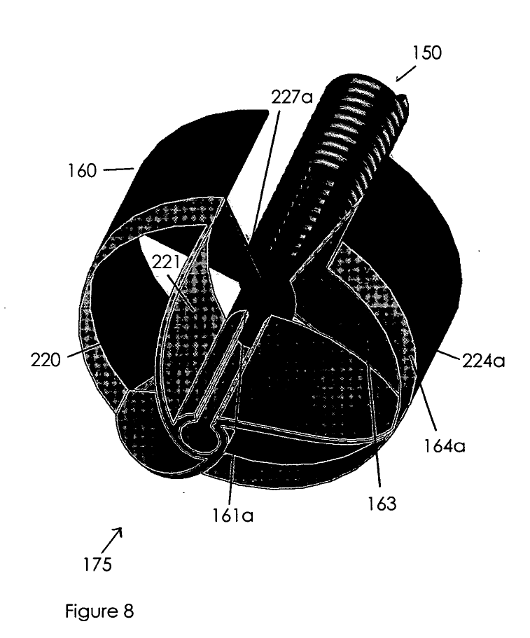

The fluid-propelling/drive assembly 170 in FIG. 7 shows the optional fluid-propelling component 160 and the internal drive shaft 150. FIG. 7 identifies the elements of the fluid-propelling component 160 including an internal cylindrical hub 161 having a distal face 161a and a proximal face 161b and containing a lumen 162 for the passage of a guide wire, or fluids, or both simultaneously, individual fins 163, and a ring housing 164 having a distal face 164a and a proximal face 164b. The preferred embodiment in FIG. 7 and FIG. 8 shows three fins, each connected between the internal cylindrical hub 161 and the ring housing 164, but the number of fins is not so limited. In this preferred embodiment, the fluid-propelling component 160 may be fabricated using similar materials and methods as those described for the internal rotational cutter 220. The drive shaft 150 may be connected to the fluid-propelling component 160 via laser welding as described for FIG. 3c.

FIG. 8 shows the fluid-propelling component 160 connected to the internal rotational cutter 220. FIG. 8 shows the internal rotational cutter 220, the fluid propelling element 160, and the internal drive shaft 150 as an assembly 175. The internal rotational cutter 220 and the fluid propelling element 160 may be attached via 1) a mating between the proximal face 224a of the internal rotational cutters annular ring 224 and the fluid-propelling elements distal face 164a, and 2) a mating between the proximal face 227a of the central hub of the internal rotational cutter and the distal face 161a of the internal cylindrical hub 161 of the fluid-propelling element. These components may be mated using laser welding as previously described. Further, in the preferred embodiment each fin 163 may be aligned with one of the rotational cutting blades 221 of the internal rotational cutter 220. The alignment of each fin 163 with a corresponding cutting blade 221 allows open spaces between cutting blade/fin combinations.

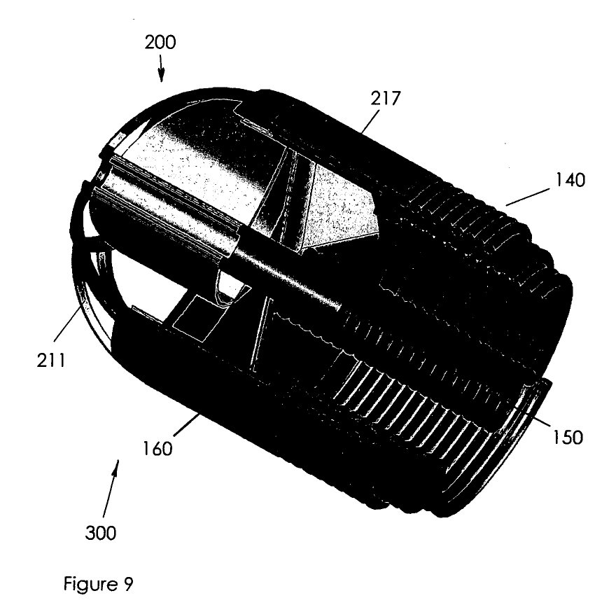

FIG. 9 shows a preferred embodiment 300 of the catheter system that employs the fluid-propelling element 160, and also shows the distal housing 211, the collar housing 217, the internal cutter 220, the catheter shaft 140, and the internal drive shaft 150. As vascular plaque material is shaved within the distal housing 211 it will become suspended within the infused saline solution and the fluid-propelling component 160 will serve to quickly remove this fluid suspension from the interior of the distal housing 120 and into the annular space between the catheter shaft 140 and the drive shaft 150. Referring to the preferred embodiment in FIG. 8, if the fluid-propelling component 160 is rotated counter-clockwise in a continuous fashion, as viewed proximal to distal along the catheter central axis, fluid within the distal housing will be urged to flow in a proximal direction within the annular space between the drive shaft 150, and the inner surface of the catheter shaft 140. Alternatively, the rotation of the drive shaft 150 may be alternated counter-clockwise / clockwise such that the fluid within the annular space between the drive shaft 150, and the inner surface of the catheter shaft 140 undergoes a oscillatory or cyclic motion, imparting a back-and-forth movement of fluid within the distal housing 110. The purpose of the back-and-forth fluid movement is to prevent particles of plaque within solution inside the catheter from being caught within any of the features of the housing or the rotating components. Additionally, it is preferred that the counter-clockwise rotation be of a longer duration than the clockwise rotation such that the net effect is to move the fluid suspension in a proximal direction through the catheter shaft, and to exit the catheters proximal port. The pitch of each fin 163, that is, the angle between the plane of the fin 163 and the central axis of the hub 161 may range ideally between 30 and 60 degrees, but is not so limited. The speed of rotation of the drive shaft may vary considerably, from 10 revolutions per second to 1000 revolutions per second, but is also not so limited. In general, slower revolutions may lead to relatively larger particles of plaque shaved from the vessel and lesser fluid-propelling action, and faster revolutions may lead to relatively smaller particles of plaque shaved from the vessel and greater fluid-propelling action. In each scenario of imparting rotational movement to the drive shaft 150, internal rotational cutter 220 and propelling component 160, vacuum applied at the catheters proximal port will continue to transport the saline-plaque suspension proximally until it exits the catheters proximal port and is removed from the catheter completely.

In any of the aforementioned catheter embodiments, the catheter central lumen that is useable to pass a guide wire, or fluids, or both simultaneously, may be used to insert a guide wire with a shaped distal segment. The shaped distal segment may be advanced and positioned within the distal end of the catheter. In this manner, the distal flexible segment of the catheter may take on the shape of the wire. This technique may be used by the physician to assist in guiding the catheter into or through various vascular tortuosities or curvatures. In a similar fashion, the catheter central lumen may also be used to shuttle an ultrasound catheter, ultrasound guide wire, and Doppler catheter or Optical Coherence Tomography system. The working element of these systems may be advanced just beyond the distal port of the catheters central lumen. In this way, each of these systems may be useful to provide the physician with information about the vessel that may facilitate the catheters passage through the occluded or stenosed blood vessel.

While descriptions of preferred embodiments of the invention have been provided above, various alternatives, modifications, combinations and equivalents may be used. Therefore, the above descriptions should not be taken as limiting the scope of the invention which is defined by the appended claims.

What is claimed is:

1. A catheter system for operation within a stenosed blood vessel, comprising:
a torqueable, flexible catheter shaft having at least one lumen;
a distal housing having an external surface and an internal surface, the housing including a series of openings that allow vascular plaque tissue to enter the interior of the distal housing along a vector that is parallel to the axis of the catheter shaft, the portion of the external surface including the openings so defined wherein a line touching the external surface lies tangential to the surface if the line is contained within the plane produced by the line and the catheters central axis, and wherein the distal end of the catheter shaft is coupled to the proximal end of the distal housing;
an internal rotational cutter having blades that are in proximity to the portion of the inner surface of the distal housing that includes the openings; and
an internal torqueable drive shaft coupled to the internal rotational cutter such that rotational motion applied to the drive shaft is communicated to the cutter to move the edge of the cutting blades along the portion of the inside surface of the housing that includes the openings, and in a rotational direction about the axis of the catheter.

2. The catheter system of claim 1, wherein a distal portion of the distal housing surface defines a convex shape.

3. The catheter system of claim 1, wherein a proximal portion of the distal housing surface defines a cylindrical shape.

4. The catheter system of claim 1, further comprising an internal lumen, the proximal port of which exits at the proximal portion of the catheter, and the distal port of which exits at the distal housing.

5. The catheter system of claim 5, wherein the catheter tracks over a guide wire via the internal lumen.

6. The catheter system of claim 5, wherein fluids are advanced within the catheter lumen to exit the catheter at the distal housing.

7. The catheter system of claim 1, further comprising an internal lumen in communication with the pattern of openings in the distal housing and a port at the proximal end of the catheter.

8. The catheter system of claim 1, further comprising a fluid-propelling component coupled between the drive shaft and the internal rotational cutter and rotatable along the axis of the catheter shaft, wherein rotation of the fluid-propelling component in a first direction causes fluid movement within the distal housing along a first axial direction, and wherein rotation of the fluid-propelling component in a second direction causes fluid movement in an opposite axial direction.

9. A catheter system for operation within an occluded blood vessel, comprising:
a torqueable, flexible catheter shaft having at least one lumen;
a distal housing having an external surface and an internal surface, the housing including a series of openings that allow vascular plaque tissue to enter the interior of the distal housing along a vector that is parallel to the axis of the catheter shaft, the portion of the external surface including the openings so defined wherein a line touching the external surface is tangential to the surface if the line is included within the plane produced by the line and the catheters central axis, and wherein the distal end of the catheter shaft is coupled to the proximal end of the distal housing;
an internal rotational cutter including blades that are in proximity to the portion of the inner surface of the distal housing including the openings; and
an internal torqueable drive shaft coupled to the internal rotational cutter, such that rotational motion applied to the drive shaft is communicated to the cutter to move the edge of the cutting blades along the portion of the inside surface of the housing that includes the openings, and in a rotational direction about the axis of the catheter.

10. A method of performing atherectomy of plaque tissue within a stenosed blood vessel, comprising
advancing an atherectomy catheter over a guide wire placed within an intravascular space, wherein the catheter includes at least one inner lumen, a flexible catheter shaft, and a distal housing with a pattern of openings to communicate to the interior of the distal housing;
an internal rotational cutter including cuffing blades that translate along the interior surface of the distal housing and the pattern of openings, an internal drive shaft coupled to the internal rotational cutter, and a proximal catheter port configured to translate a vacuum to within the interior of the distal housing;
advancing the distal housing against the vascular plaque wherein the plaque is engaged within the distal housing openings and impinges through the openings and into the interior of the distal housing along a vector that is parallel with the axis of the catheter shaft;
imparting a rotation to the internal drive shaft and the rotational cutter, wherein the vascular plaque tissue that has impinged to within the interior of the distal housing is shaved off by the cutting blades;
rotating the catheter to translate the distal housing openings to previously uncut portions of the vascular plaque, and repeating the process of shaving the plaque off within the interior of the distal housing;
advancing the catheter forward over the guide wire and into the space created by the vascular plaque removal; and
removing the catheter, leaving the guide wire in place in the vessel.

11. The method of claim 10, wherein the pattern of openings in the distal housing are arranged along a convex contour of the distal housing.

12. The method of claim 11, wherein the vascular plaque tissue is engaged through the openings in the distal housing along a vector that is parallel with the axis of the catheter shaft.

13. A method of creating a patent pathway through a vascular total occlusion comprising:
advancing an atherectomy catheter within an intravascular space, wherein the catheter includes at least one inner lumen, a flexible catheter shaft, a distal housing with a pattern of openings to communicate to the interior of the distal housing, an internal rotational cutter including cutting blades that translate along the interior surface of the distal housing and the pattern of openings, an internal drive shaft coupled to the internal rotational cutter, and a proximal catheter port configured to translate a vacuum to within the interior of the distal housing;
advancing the distal housing against the vascular plaque, wherein the plaque is engaged within the distal housing openings and impinges through the openings and into the interior of the distal housing along a vector that is parallel with the axis of the catheter shaft;
imparting rotation to the internal drive shaft and the rotational cutter, wherein the vascular plaque tissue that has impinged to within the interior of the distal housing is shaved off by the cutting blades;
rotating the catheter to translate the distal housing openings to previously uncut portions of the vascular plaque, and repeating the process of shaving the plaque off within the interior of the distal housing; and
advancing the catheter forward and into the space created by the vascular plaque removal, and continuing the process of plaque shaving until the catheter has established a pathway through the vascular occlusion.

14. The method of claim 13, wherein the pattern of openings in the distal housing are arranged along a convex contour of the distal housing.

15. The method of claim 14, wherein the vascular plaque tissue is engaged through the openings in the distal housing along a vector that is parallel with the axis of the catheter shaft.

16. A catheter system for operation within a stenosed blood vessel, comprising:
a catheter shaft having at least one lumen; a convex distal housing that includes a series of openings along a convex surface that allow vascular plaque tissue to enter the interior of the distal housing;
an internal rotational cutter having blades that are in proximity to the portion of the inner surface of the distal housing that includes the openings; and
a drive shaft coupled to the internal rotational cutter.
